(19) United States
(12) Patent Application Publication
(10) Pub. No.: US 2006/0241549 A1
(43) Pub. Date: Oct. 26, 2006

(54) Apparatus and method for precise ozone/oxygen delivery applied to the treatment of dermatological conditions, including gas gangrene, and related disorders

(76) Inventor: Gerard V. Sunnen, New York, NY (US)

Correspondence Address: OSTROLENK FABER GERB & SOFFEN, 1180 AVENUE OF THE AMERICAS, NEW YORK, NY, 100368403, US

(21) Appl. No.: 11/110,066

(22) Filed: Apr. 20, 2005

(51) Int. Cl. A61M 37/00

(52) U.S. Cl. 604/23

(57) ABSTRACT

An apparatus and a method for the therapy of a range of dermatological conditions using precisely formulated gaseous milieus featuring the beneficial functions of oxygen and ozone. A range of clinical applications for this system include the therapy of gas gangrene, infected wounds, poorly healing wounds, war wounds, decubitus ulcers, dermatological conditions due to circulatory disorders, lymphatic diseases of the skin, fungal skin infections, burns, nail afflictions, radiodermatitis, parasitic skin infestations, and frostbite.

### BACKGROUND OF THE INVENTION

1. Field of the Invention

The present invention relates to an apparatus and method for precise ozone/oxygen delivery applied to the treatment of dermatological conditions, including gas gangrene, and related disorders.

2. Related Art

Ozone

Ozone, in its gaseous form, provides superb antipathogenic action for a wide range of bacteria, viruses, protozoa, and parasites. Furthermore, ozone, in appropriately administered concentrations, possesses physiological properties capable of enhancing the healing of tissues.

Ozone, an allotropic form of oxygen, possesses unique properties which are being defined and applied to biological systems as well as to clinical practice. As a molecule containing a large excess of energy, ozone, through yet incompletely understood mechanisms, manifests bactericidal, virucidal, and fungicidal actions which may make it a treatment of choice in certain conditions and an adjunct to treatment in others. The oxygen atom exists in nature in several forms: (1) As a free atomic particle (O), it is highly reactive and unstable. (2) Oxygen (O₂), its most common and stable form, is colorless as a gas and pale blue as a liquid. (3) Ozone (O₃), has a molecular weight of 48, a density one and a half times that of oxygen, and contains a large excess of energy in its molecule (O₃ [symbol] 3/2 O₂+143 KJ/mole). It has a bond angle of 127±3, is magnetic, resonates among several forms, is distinctly blue as a gas, and dark blue as a solid. (4) O₄ is a very unstable, rare, nonmagnetic pale blue gas, which readily breaks down into two molecules of oxygen.

Ozone is a powerful oxidant, surpassed in this regard only by fluorine. Exposing ozone to organic molecules containing double or triple bonds yields many complex and as yet incompletely configured transitional compounds (i.e. zwitterions, molozonides, cyclic ozonides), which may be hydrolysed, oxidized, reduced, or thermally decomposed to a variety of substances, chiefly aldehydes, ketones, acids, and alcohols. Ozone also reacts with saturated hydrocarbons, amines, sulthydryl groups, and aromatic compounds.

Importantly relevant to biological systems is ozone's interaction with tissue constituents including blood. The most studied is lipid peroxidation, although interactions have yet to be more fully investigated with complex carbohydrates, proteins, glycoproteins, and sphingolipids.

These properties are responsible for ozone's ability to destroy a wide spectrum of pathogens.

The Effects of Ozone on Pathogens

Infected wounds, and especially chronic lesions, may show a wide spectrum of profuse pathogen growth, including bacteria, viruses, fungi, and protozoa.

The anti-pathogenic effects of ozone have been substantiated for several decades. Its panpathogen properties are universally recognized and serve as the basis for its increasing use in disinfecting municipal water supplies in cities worldwide.

Bacteria

Indicator bacteria in effluents, namely coliforms and pathogens such as Salmonella, show marked sensitivity to ozone inactivation. Other bacterial organisms susceptible to ozone's disinfecting properties include Streptococci, Staphylococci, Shigella, Legionella, Pseudomonas, Yersinia, Campylobacter, Mycobacteria, Klebsiella, and Escherichia coli.

Ozone destroys both aerobic, and importantly, anaerobic bacteria, which are mostly responsible for the devastating sequelae of complicated infections, as exemplified by decubitus ulcers and gangrene.

The mechanisms of ozone bacterial destruction need to be further elucidated. It is known that the cell envelopes of bacteria are made of polysaccharides and proteins, and that in Gram-negative organisms, fatty acid alkyl chains and helical lipoproteins are present. In acid-fast bacteria, such as Mycobacterium tuberculosis, one third to one half of the capsule is formed of complex lipids (esterified mycolic acid, in addition to normal fatty acids), and glycolipids (sulfolipids, lipopolysaccharides, mycosides, trehalose mycolates).

The high lipid content of the cell walls of these ubiquitous bacteria may explain their sensitivity, and eventual demise, in the face of ozone exposure. Ozone may also penetrate the cellular envelope, directly affecting cytoplasmic integrity.

Viruses

Numerous families of viruses including poliovirus 1 and 2, human rotaviruses, Norwalk virus, Parvoviruses, and Hepatitis B and C, among many others, are susceptible to the virucidal actions of ozone.

Most research efforts on ozone's virucidal effects have centered upon ozone's propensity to splice lipid molecules at sites of viral multiple bond configuration. Indeed, once the lipid envelope of the virus is fragmented, its DNA or RNA core cannot survive.

Non-enveloped viruses (Adenoviridae, Picornaviridae (poliovirus), Coxsachie, Echovirus, Rhinovirus, Hepatitis A, D, and E, and Reoviridae (Rotavirus), have also been studied in relation to ozone inactivation. Viruses that do not have an envelope are called “naked viruses.” They are constituted of a nucleic acid core (made of DNA or RNA) and a nucleic acid coat, or capsid, made of protein. Ozone, in addition to its well-recognized action upon unsaturated lipids, can interact with certain viral proteins and amino acids. Indeed, when ozone comes in contact with capsid proteins, protein hydroxides and protein hydroperoxides are formed.

Viruses have no protection against oxidative stress. Normal mammalian cells, on the other hand, possess complex systems of enzymes (e.g., superoxide dismutase, catalase, peroxidase) which tend to ward off the nefarious effects of free radical species and oxidative challenge. It may thus be possible to treat infected tissues with ozone while respecting the integrity of their healthy cell components.

Herpes viruses are widespread in the human population. Two distinct types of viruses are known, Herpes simplex type I and II, both lipid-enveloped. Type I is transmitted via contact through the mucosa or broken skin (often through saliva), while type II is sexually propagated.

Herpes lesions have been extensively studied with reference to topical ozone administration. Ozone (1) directly inactivates herpes viruses that are lipid-enveloped, (2) acts as a pan-bactericidal agent in cases involving secondary infections, and (3) promotes healing of tissues through circulatory enhancement.

Fungi

Fungi families inhibited and destroyed by exposure to ozone include Candida, Aspergilus, Histoplasma, Actinornycoses, and Cryptococcus. The cell walls of fungi are multilayered and are composed of approximately 80% carbohydrates and 10% of proteins and glycoproteins. The presence of many disulfide bonds has been noted, making this a possible site for oxidative inactivation by ozone.

Protozoa

Protozoan organisms disrupted by ozone include Giardia, Cryptosporidium, and free-living amoebas, namely Acanthamoeba, Hartmonella, and Negleria. The exact mechanism through which ozone exerts anti-protozoal action has yet to be elucidated.

Cutaneous Physiological Effects of Ozone/Oxygen

The positive effects of oxygenation on many dermatological conditions have long been established, and form the basis for the use of hyperbaric oxygen treatment. Oxygen diffuses into the tissues, raising their oxidation-reduction potential, thus inhibiting the growth of anaerobic bacteria.

Ozone greatly supplements the benefits of oxygen administration alone. While the most likely beneficial effect of external ozone administration is pathogen inactivation, it is important to note ozone's contribution to healing through its physiological actions. Ozone dilates the arterioles in wounds, thus stimulating the inflow of nutrients and immunological molecules. By similar mechanisms, the outflow of waste products is accelerated.

Medical Conditions Benefited by Ozone Therapy

In view of the above-mentioned principles of ozone/oxygen's biological properties, the present invention seeks to harness this therapeutic potential, not only for the treatment of several dermatological conditions, but also for their prevention.

The following is a list of pathologic sequelae of tissue compromise which may be addressed by external ozone/oxygen therapy. The most serious is gangrene, and the most ominous is gas gangrene.

Gas Gangrene

Gas gangrene may be a rapidly fatal complication of traumatic injuries such as automobile accidents and war injuries, surgical incisions, wounds, burns, and decubitus ulcers, among many other conditions. Predisposing factors include diabetes, arteriosclerosis, lesions associated with colon cancer, surgeries involving the intestinal tract, and septic abortions.

Gas gangrene, also known as necrotizing fascitis, myositis, and myonecrosis is feared because of the rapidity of its evolution and the galloping and irreversible demise of affected tissues.

Several bacterial species are implicated in this process, the most common being Clostridium families. These anaerobic bacteria thrive in the absence of oxygen, feeding on glycogen and sugars, producing lactic acid, and gases such as methane, carbon dioxide, and hydrogen, among others. They also produce toxins causing hemolysis, renal failure, shock, coma, and death, as they are diffused systemically.

Other bacterial species are implicated in gas gangrene aside from Clostridium, including Enterobacteria, E. coli, Proteus, Group A streptococcus, Staphylococcus, Vibrio, Bacteriodes, and Fusiforms. Ozone is effective in inactivating all of these anaerobes and aerobes.

The proposed invention aims at the early detection of the onset of gas gangrene in wounds that are clinically deemed to be potentially at risk, and for early therapeutic responses via calibrated ozone/oxygen infusion.

This is achieved by means of the intra-envelope bacterial gas sensor providing a warning of gas buildup, including, but not limited to, methane, hydrogen, carbon dioxide, indoles, and skatoles, and by the automatic commensurate response through microprocessor-mediated ozone/oxygen infusion into the treatment envelope, at a concentration and for a duration predicated upon programmed treatment protocols.

Infected Wounds

This category of wound has, by definition, not yet reached the status of chronicity due to a combination of circulatory compromise and infective onslaught. In fact, this category of wound may simply be post-surgical, and only potentially prone to infection.

The use of topical ozone therapy in these cases may be solely preventive, aimed at improving circulation on one hand, and inhibiting the proliferation of potentially infective organisms on the other.

Poorly Healing Wounds

Wounds which heal in an indolent manner are frustratingly difficult to master. Generally speaking, poorly healing wounds owe their definition to their chronicity, which is most commonly caused by the profusion and variety of offending organisms they harbor.

War Wounds

War wounds often present complex treatment challenges. Compound fractures are common. Healing is often complicated by the presence of shrapnel and other foreign bodies. Infection is favored by hot weather and high humidity.

Ozone/oxygen external application offer excellent prophylaxis for the infectious processes made likely by the special nature of war wounds.

Decubitus Ulcer

This common condition arises when a patient remains in bed, or in a wheelchair, in a restricted position for a prolonged period of time. The pressure exerted upon skin contact points compresses the dermal arterioles preventing the proper perfusion of tissues. This leads to tissue oxygen starvation, impaired skin resilience, and the eventual breakdown of the skin itself. An expanding ulcer develops, usually infected by a spectrum of pathogenic organisms. At times the breakdown is so severe that the ulcer reaches the bone, ushering in osteomyelitis.

The treatment of decubitus ulcers requires a multidisciplinary approach, including surgical, pharmacological, and physiological interventions. Topical antibiotics often fail to penetrate the depth of the wound, are active only against a limited spectrum of organisms, induce resistance, and not infrequently cause secondary dermatitis in their own right.

Aside from the benefits of topical ozone therapy described in this text, it should be mentioned that an added therapeutic feature of ozone, especially as it relates to the treatment of deep ulcers, is its capability to penetrate into deeper tissue levels, thereby affecting pathogens which would normally be protected by tissue overlay.

Circulatory Disorders

This class of disorders has one common denominator, namely impaired circulation to tissues via compromise of vascular integrity. A prototypic disease is diabetes. Diabetes manifests vascular disturbances to many organ systems (e.g. retina, kidney), and concomitant disruptions to carbohydrate metabolism. In cases where diabetes affects the peripheral circulation, tissues such as the dermis become vascularly compromised, and thus more prone to injuries and infections.

Diabetic ulcers frequently develop following abrasions, contusions, and pressure injuries. These ulcers, not unlike decubitus ulcers, are notoriously difficult to treat. Topical ointments can only address a minor spectrum of putative infectious organisms. These same organisms, furthermore, may rapidly develop antibiotic resistance.

Serially applied ozone topical therapy inactivates most, if not all, offending pathogens and these same pathogens are unable to build a resistance to its effects.

Arteriosclerosis is a condition marked by the thickening and hardening of the vascular tree. The normal pliability and patency of blood vessels is compromised, leading to impaired circulation in many organ systems. In the face of reduced peripheral circulation (e.g., arteriosclerosis obliterans), skin disorders may include trophic changes (e.g., dry hair, shiny skin) apt to injury and eventual ulcer formation.

Lymphatic Diseases

The lymphatic system regulates fluid equilibration within the body and, most importantly, offers infection defense.

Lymphedema is a condition caused by blockage to lymphatic drainage. It may be secondary to trauma, surgical procedures, and infections (e.g., streptococcal cellulitis, filiriasis, lymphogranuloma venereum).

Increasingly common is lymphedema resulting from surgical removal of lymph nodes following surgery for breast cancer. The affected arm in these patients is likely to be chronically swollen and indurated. Exercises are routinely prescribed to develop collateral circulation. Most alarming, however, is the occurrence of infections following even minor injuries to the arm. Injuries are then much more likely to become infected due to the absence of lymphatic system defenses. In these cases, intensive topical wound care is initiated, and systemic antibiotic treatment is prescribed.

Topical ozone treatment applied in a timely fashion to the affected hand or arm may prevent secondary infection; and, it may avoid the need for systemic antibiotics.

Fungal Skin Infections

Fungi are present on human skin in a quasi-symbiotic relationship. Candida, Aspergillus, and Histoplasma, for example, are often found on intact skin, without causing clinical problems.

However, under certain conditions, the normal balance of the dermis is disturbed, allowing superficial fungi to proliferate. Tinea capitis is manifested by pustular eruptions of the scalp, with scaling and bald patches. Tinea cruris is a fungal pruritic dermatitis in the inguinal region.

Serial topical ozone applications have shown marked success in eradicating the most chronic and stubborn fungal skin conditions.

Burns

Thermal burns are divided into first, second, and third degrees, depending upon the depth of tissue damage. First-degree burns are superficial, and include erythema, swelling, and pain. In second degree burns, the epidermis and some portion of the underlying dermis are damaged, leading to blister and ulcer formation. Healing occurs in one to three weeks, usually leading to little or no scar formation.

In third degree burns, muscle tissue and bone may be involved, and secondary infection is common.

It is in cases marked by significant tissue injury, and especially in cases involving infections, that topical ozone therapy finds the most usefulness. In the case of burns, the spectrum of pathogenic organisms may be wide and thus may be ideally suited for ozone therapy.

In burns, externally applied ozone concentrations need to be carefully calibrated. The clinician must be able to gauge the proper ozone concentration geared to the specific medical condition under treatment. In wet burns, for example, initial ozone concentrations will need to be low, in order to prevent inordinate systemic absorption through absorption of exudates. As the burn heals and progressively dries, greater ozone concentrations may then be administered.

Nail Afflictions

Conditions implicating nails which are therapeutically assisted by topical ozone treatment include the following:

1. Candida albicans. Nails in this condition are painful, with swelling of the nail fold, and often, thickening and transverse grooving of the nail architecture. Loss of the nail itself may occur. Another frequent condition is Tinea Unguium, marked by thickened, hypertrophic, and dystrophic toenails. There are currently no topical antifungal agents of proven efficacy for this condition. Systemic anti-fungal agents show a spectrum of noxious side effects.

2. Tinea Pedis (Athlete's Foot). This very common disorder is caused by infection with species of Trichophyton, and with Epidermophyton floccosum. Chronic infection involving the webbing of the toes may evolve to secondary bacterial involvement. Lymphangitis and lymphadenitis may present themselves, as well as infection of the nails themselves (Tinea Unguium; Onychomycosis). Nails may become thickened, yellow, and brittle. The patient may then develop allergic hypersensitivity to these organisms.

Topical ozone therapy offers unique treatment opportunities to these recalcitrant infections. Ozone penetrates the affected areas, including the nails proper, and with repeated administration, is capable of inactivating all species of fungi mentioned above. Healing occurs slowly yet consistently, and skin integrity along with nail anatomy, gradually regain their normal configuration.

Radiodermatitis

This condition occurs during times when the body is exposed to ionizing radiation. This may result from radiological accidents or from radiation therapy. Radiation energy, imparted to cells, leads to cellular DNA injury.

Clinical findings are proportional to the type, amount, and duration of radiation exposure. Several clinical syndromes have been delineated, including Radiation Erythema and Radiodermatitis.

While DNA damage cannot be easily repaired, secondary infections made more likely by decreased tissue resistance may be countered by topical ozone therapy. This avoids the systemic absorption of topical ointments and provides pan-pathogen protection.

Frostbite

Factors contributing to skin injuries due to cold derive from vasoconstriction and the formation of ice crystals within tissues. As frostbite progresses, loss of sensation occurs, and tissues become increasingly indurated to touch. Depending upon length of exposure, dry gangrene may develop. Dry gangrene may then evolve to wet gangrene if infection occurs.

Topical oxygen/ozone therapy has proven to be effective in decelerating or halting the pathogenesis of frostbite through (1) immediate oxygenation of tissues, (2) increasing blood flow through a direct vasodilatory effect upon the dermal arterioles, and (3) prevention of secondary infection.

The present invention allows a microprocessor-controlled intra-envelope milieu geared to the therapy of frostbite, including proper temperature, humidity, and appropriate ozone/oxygen concentrations.

Advantages of Topical Ozone Therapy

Topical ozone/oxygen therapy for the disorders mentioned above requires diagnosis of the underlying conditions, and a correspondingly appropriately tailored treatment plan, which may include any one of several therapeutic modalities utilized concomitantly, including ozone, or may call for the utilization of ozone as the sole therapeutic intervention.

The salient advantages of topical ozone/oxygen therapy include:
1. The ease of administration of this therapy.
2. Ozone is an effective antagonist to the viability of an enormous range of pathogenic organisms. In this regard, ozone cannot be equaled. It is effective in inactivating anaerobic and aerobic bacterial organisms and a wide swath of viral families—lipid as well as non-lipid enveloped—and fungal and protozoan pathogens. To replicate this therapeutic action, the medical conditions in question would have to be treated with complex conglomerations of antibiotic agents.
3. Ozone/oxygen therapy, appropriately applied in a timely fashion, may obviate the need for systemic anti-pathogen therapy, thus saving the patient from the side effects this option could entail.
4. Ozone exerts its anti-pan-pathogenic actions through entirely different mechanisms than conventional antibiotic agents. The latter must be constantly upgraded to surmount pathogen resistance and mutational defenses. Ozone, on the other hand, presents direct oxidative challenge which cannot be circumvented by known mechanisms of pathogen resistance.

### SUMMARY OF THE INVENTION

In view of the foregoing benefits of ozone/oxygen treatments, the present invention provides an apparatus and method for precise ozone/oxygen delivery applied to the treatment of dermatological conditions, including gas gangrene, and related disorders.

While drugs administered in solid or liquid form are easily quantifiable, drugs in gaseous form present special dosing difficulties, namely the accurate measurement of gas concentration as a function of time of exposure, temperature, and humidity content. Others may need more modulated treatments. In other scenarios, some lesions, in their acute states, may initially require certain dosage administrations, while later in the course of the same treatment session, the required dosage may change.

This invention addresses the vital importance of the effective dosing of ozone/oxygen mixtures to the therapy of acutely and chronically infected dermatological lesions. Indeed, without correct dosing of any therapeutic agent, proper medicine cannot be practiced.

The therapeutic action of gaseous ozone/oxygen mixtures derives from the antipathogenic effects, and the beneficial physiological effects, of both ozone and oxygen. However, to be optimally effective, ozone/oxygen mixtures applied to the spectrum of dermatological pathologies must be carefully calibrated.

This invention embodies an ozone delivery system specifically aimed at the treatment of skin pathologies. As such, it is a dermatological ozone/oxygen delivery system.

Therapeutic ozone/oxygen mixtures applied to external wounds or other dermatological conditions have, to this day, been administered in an imprecise fashion at best. The essential requirement of precise dosing to the rigorous demands of scientific research and to clinical practice has consequently been hampered by this shortcoming.

Externally administered ozone/oxygen mixtures have been applied to the treatment of dermatological conditions since before World War One. The German armed forces fashioned rubber envelopes to surround and seal injured limbs and circulated ozone/oxygen mixtures within them. These mixtures were delivered by field generators because ozone reverts relatively rapidly to oxygen at room temperature, and cannot be stored except at very low temperatures.

Unfortunately, these rubber envelopes frittered easily due to ozone's high oxidative power. Modern materials are available, such as plastics and silicones, that are impervious to oxidation.

A previous invention (Sunnen, U.S. Pat. No. 6,073,627) included a transparent envelope with inserted sensors for ozone concentration, humidity, and patient temperature located within the treatment envelope, each relaying data to a display on the ozone generator panel.

Previous art such as U.S. Pat. No. 6,073,627 attempted to address this issue. This art described an ozone generator which delivered an ozone/oxygen mixture into a treatment envelope encasing the patient's lesion. The problem of delivering a precise ozone/oxygen mixture, however, was only partially solved by this art, based upon the following considerations:
1. The ozone concentration within the treatment envelope was relayed to a readout gauge on the ozone generator, to be read by the clinical personnel. In order to maintain a constant ozone concentration over time and thus adhere to a precise treatment protocol, the personnel would be obliged not only to be present during the entire treatment process but also to adjust the generator's output in response to the fluctuations normally observed in intra-envelope ozone concentrations.
It would therefore be desirable to have a delivery system with an automatic microprocessor-mediated feedback of intra-envelope ozone concentrations in order to counteract their fluctuations in a timely fashion.
2. The temperature of the patient was monitored, but the temperature inside the treatment envelope was not. Intra-envelope ambient temperature is an integral part of the treatment protocol of external wounds with ozone/oxygen. Indeed, some dermatological lesions, such as frostbite, require higher therapeutic ambient temperatures while others do not. Furthermore, temperature itself has an influence upon ozone concentration, with lower temperatures associated with higher concentrations.
It would therefore be desirable to provide a delivery system with a constant integration of ozone and temperature and an automatic microprocessor-mediated feedback of intra-envelope temperature to achieve temperature-to-ozone constancy.
3. Intra-envelope humidity influences ozone concentration, with higher ozone output by the generator needed at higher humidity levels to maintain a constant ozone concentration. The therapy of dermatological conditions requires attention to the maintenance of intra-envelope humidity levels. Some lesions, such as wet gangrene, must be kept dry, while others need moisture. This invention may comprise an automatic microprocessor-mediated regulation of humidity levels to achieve constancy of the intra-envelope humidity milieu.
4. The space within the treatment envelope can show significant regional variations and fluctuations in ozone concentration, temperature, and humidity, depending upon the placement of probes and the unavoidable presence of pockets of “dead space.” This invention may comprise an intra-envelope fan to homogenize the ambient ozone/oxygen mixture so that probe readings will be accurate.
5. Treatment envelopes in U.S. Pat. No. 6,073,627 were mere plastic bags. They required careful adjustment to anatomical parts so as to minimize unnecessary dead space, offer patient convenience, and avoid apposition of the envelope sheath to the patient's tissues. Described herein are improved envelopes having rigid or flexible supporting ribs or other supporting structures which address these needs.

The present invention may comprise a further addition, namely a sensor inserted in the treatment envelope capable of detecting gases emitted by pathogenic bacteria growing in the wounds under treatment. These gases are typically observed in gangrenous conditions, including gas gangrene. It is of paramount importance to possess early warning of the development of gangrene, because this condition may evolve so rapidly that the patient's life can be saved only by early amputation. In addition to the early detection of gangrene, this invention addresses the early preventive treatment of this potentially fatal sequel of surgical wounds, war wounds, decubitus ulcers, burns, and traumatic injuries.

The present invention therefore may comprise a microbial gas sensor to monitor the bacterial activity in the wound under treatment. The presence and concentration of pathogen-generated gases are relayed to the generator which, via microprocessor-mediated feedback, modifies the envelope milieu and the duration of the treatment.

Microprocessor-mediated feedback allows the ozone concentration, the humidity, and the temperature within the treatment envelope to be automatically maintained at predetermined and constant levels, if so chosen, or alternatively, to respond to the changing parameters of the wounds under treatment. The sensors within the envelope may thus provide feedback data to modify:
1. The generator's output of ozone concentration via the automatic regulation of oxygen flow through the system and/or the regulation of electrical or other energy applied to the medical grade oxygen for conversion to ozone.
2. The generator's humidity control to satisfy the treatment's humidity requirement.
3. The generator's heat control output.

The automatic feedback-mediated adjustment of these parameters is important because it avoids the need for the clinician's constant monitoring of the treatment process. Since treatment duration times range anywhere from a few minutes to several hours or more, it is cumbersome to oversee and hand-regulate delivery system functions in response to the readings of envelope sensors. Such adjustments are not only cumbersome; they make for significant dosage inaccuracies over the range of the treatment session.

The treatment session may be further automated by means of a timer which may (1) shut off ozone delivery to the envelope once the predetermined treatment time has elapsed; (2) shut off ozone delivery to the envelope once the bacterial gas sensors have signaled to do so; or (3) withdraw ozone/oxygen from the envelope while simultaneously infusing it with oxygen, thus signaling the termination of the treatment process.

The treating personnel may then remove the envelope at some time after the treatment cycle is completed. The advantage of this automated process lies in the fact that precise termination of treatment is not predicated upon the constant presence of treatment staff.

Methods of Ozone Generation and Administration

Ozone generation and delivery systems utilized for human and veterinary purposes require that ozone be generated at the time it is to be administered. Ozone is not a drug that has a substantial shelf life. Indeed, as a gas, ozone has a half-life of approximately 50 minutes at room temperature.

In the case of external application, the generator supplies a dosage of ozone/oxygen determined by the clinician. This, in practice, may involve an infected foot, a diabetic ulcer, a poorly healing traumatic wound, or a war wound.

In the practice of external ozone application, a specially-designed envelope of polyester, silicone, or another ozone-inert material is utilized to encase the body part under treatment. A precise selection and/or fitting of the bag is desirable to provide (1) a proper containment of ozone/oxygen surrounding the affected area with the requirement of preventing ozone escape to the ambient air; (2) a consistent intra-envelope concentration of O₃/O₂ that will resist mixing with outside air; and (3) a comfortable fit for the patient.

In order to allow for proper circulation through the system, an exit catheter channels intra-envelope ozone/oxygen through an outlet valve 21 back to the generator 7 for catalytic re-conversion to oxygen by an ozone destructor 23.

Externally applied ozone concentrations should be carefully adjusted. The clinician must be able to gauge the proper ozone concentration geared to the specific medical condition at hand. In extensive wet burns where there are exudates, for example, initial ozone concentration may need to be relatively low in order to prevent undue systemic ozone absorption. As the burn heals and progressively dries, greater ozone concentrations may then be administered to adapt to the healing process.

In the treatment of purulent diabetic ulcers, high initial ozone doses may be needed at the beginning of the course of treatment in order to maximally reduce pus formation. Then, as fragile granulation appears, lower doses may be substituted.

The Treatment Envelope

The treatment envelope described herein provides a significant improvement over the one described in U.S. Pat. No. 6,073,627.

The envelope described in that application is flaccid and thus has the inconvenience of coming in contact with the lesions being treated when it is not inflated.

The present invention embodies a rigid or flexible armature (such as ribs) providing supportive architecture to the treatment envelope in such a manner as to permit a limb to be placed within it without the skin coming in contact with its sheath.

The envisioned armature, made of ozone-resistant material, is constructed so that it enables the affected limb to be easily inserted into the treatment envelope without contact between the envelope and the lesion. Concomitantly, it allows the space within the envelope, when fully expanded with an ozone/oxygen mixture, to attain a volume that is maximally efficient in terms of intra-envelope gas dynamics. This is important because too large a volume in the envelope at the start of treatment signifies “dead space” surrounding the limb. This dead space should be kept at a minimum, for example 10-15% of the total envelope volume, in order to prevent excessive dilution of incoming ozone/oxygen.

The treatment envelope may have any number of different configurations to adapt to the clinical situation at hand. Envisioned are the following configurations:
1. Tubular configurations adapted to the treatment of arms and legs.
2. Cylindrical configurations adapted to overlie lesions on flat surfaces of the back and abdomen.
3. Special configurations, as for example, envelopes designed in the form of briefs or shorts, for the treatment of genital or anal lesions (e.g., hemorrhoids, anal fissure, proctitis); and envelopes fitting over the scalp for the treatment of scalp infections, and infestations such as head lice.

The envelope may contain an opening through which a multi-sensor head can be easily inserted and removed. This multi-sensor head may contain sensors for:
(1) The ozone concentration
(2) The oxygen concentration
(3) The intra-envelope temperature
(4) The intra-envelope humidity, and
(5) The intra-envelope bacterial gases.

These sensors signal their readings to their respective analyzers.

In the case of the ozone concentration, the envelope ozone sensor readings are relayed to the ozone analyzer which signals the microprocessor. The microprocessor, in turn, regulates the power unit, providing compensatory ozone concentration adjustments into the envelope. To adjust ozone concentration, the microprocessor may adjust ozone generation, ozone delivery or oxygen delivery, or more than one of these factors.

The bacterial gas sensor detects accumulations of bacterial gaseous by-products and signals the generator to modify its output, as a function not only of ozone concentration, but also as a function of treatment duration. When the gas sensors detect a declining concentration of bacterial gases, for example, the generator may, depending upon protocol parameters, gradually reduce intra-envelope ozone concentration, and commensurately lengthen total treatment time.

Similar mechanisms are operational with reference to the temperature sensor, the humidity sensor, and the bacterial gas sensor.

The envelope is sealed to the skin as hermetically as possible. The escape of ozone into the milieu of the treatment space is thus minimized. This may be accomplished by any one of a number of sealing methods including Velcro-type fasteners and adhesive seals.

The treatment envelope may contain a fan which mixes the intra-envelope milieu during treatment for its homogeneous distribution. This fan may exist as a separate entity, or may be incorporated into the ozone/oxygen entry port.

According to one aspect of the invention, a dermatological ozone/oxygen treatment system is designed to provide calibrated ozone/oxygen gas mixtures to treat a wide range of dermatological pathologies; and, importantly, to provide for the automatic regulation of the therapeutic gas milieu throughout the duration of individual treatment via ongoing feedback monitoring. The delivery system comprises (1) an ozone generator, and (2) treatment envelopes designed specifically to accommodate the individual needs of patients. The ozone generator may comprise:
A. A microprocessor equipped with the capacity to evaluate incoming data from various gas analyzers, and to respond in a timely corrective fashion, in accordance with treatment protocols.
B. The analyzers process incoming data from the treatment envelope. Analyzers in the generator may include (1) the ozone analyzer (2) the oxygen analyzer, (3) the temperature analyzer, (4) the humidity analyzer, and (5) the bacterial gas analyzer.
C. The microprocessor influences the functioning of (1) the power unit, (2) the heater/cooler, and (3) the humidifier. Ozone concentration output, temperature, and humidity may thus be automatically modulated according to the changing conditions within the treatment envelope.
D. The microprocessor processes measurements from the bacterial sensor and, according to treatment protocol parameters, regulates both the ozone concentration via generator power modulation, and the length of treatment by modulating timer functions.
   2. The treatment envelope, made of variably flexible transparent ozone-resistant material, as for example, but not limited to Teflon or silicones, comprises:
A. Shapes designed to conform to the anatomical needs of the patient. Described are tubular shapes to accommodate limbs, cylindrical shapes to address lesions on flat body surfaces such as the back and abdomen, and briefs for use with anal and genital conditions (e.g., hemorrhoids, anal fissure, proctitis); also, envelopes designed to fit around the scalp for the treatment of infections involving the head, and for scalp infestations, such as with head lice.
B. The incorporation of supporting ribs or other supporting structures allowing the affected limb to stay clear of the envelope membrane and to minimize intra-envelope dead space.
C. A port allowing for the easy insertion and removal of a multi-sensor head which contains sensors for the constant analysis of the intra-envelope gas milieu.
D. The multi-sensor head contains sensors which may include, but are not limited to: (1) an ozone concentration sensor, (2) an oxygen concentration sensor, (3) a temperature sensor, (4) a humidity sensor, and (5) a bacterial gas sensor.
E. An intra-envelope fan to promote the homogeneous distribution of intra-envelope conditions.
   3. Other aspects of the invention relate to the following:
A. A dermatological ozone/oxygen delivery system designed to provide calibrated ozone/oxygen gas mixtures to treat a wide range of dermatological pathologies. Described is a range of clinical applications for this system including the therapy of gas gangrene, infected wounds, poorly healing wounds, war wounds, decubitus ulcers, dermatological conditions due to circulatory disorders, lymphatic diseases of the skin, fungal skin infections, burns, nail afflictions, radiodermatitis, and frostbite.
B. A dermatological ozone/oxygen delivery system designed to maintain a constant treatment milieu to the lesion under treatment predicated upon treatment protocol requirements. The system achieves this milieu constancy via ongoing feedback and response between intra-envelope conditions of ozone concentration, temperature, and humidity on the one hand, and central generator functions on the other.
C. A dermatological ozone/oxygen delivery system with a bacterial gas sensor to detect the emergence of gaseous by-products of pathogenic bacteria, including, but not limited, to bacteria associated with gas gangrene. This system has the capacity to regulate ozone concentration, temperature, humidity, and duration of treatment, as a function of the gases it detects.

Other features and advantages of the present invention will become apparent from the following description of embodiments of the invention which refers to the accompanying drawings.

### BRIEF DESCRIPTION OF THE DRAWINGS

FIG. 1 shows schematically the configuration of apparatus according to an embodiment of the invention, and its use for a system for external O₃/O₂ treatment of an infected leg.

FIG. 2 shows the infected leg and the treatment envelope in more detail.

FIG. 3 shows another example of a treatment envelope, for the patient's midsection.

### DETAILED DESCRIPTION OF EMBODIMENTS OF THE INVENTION

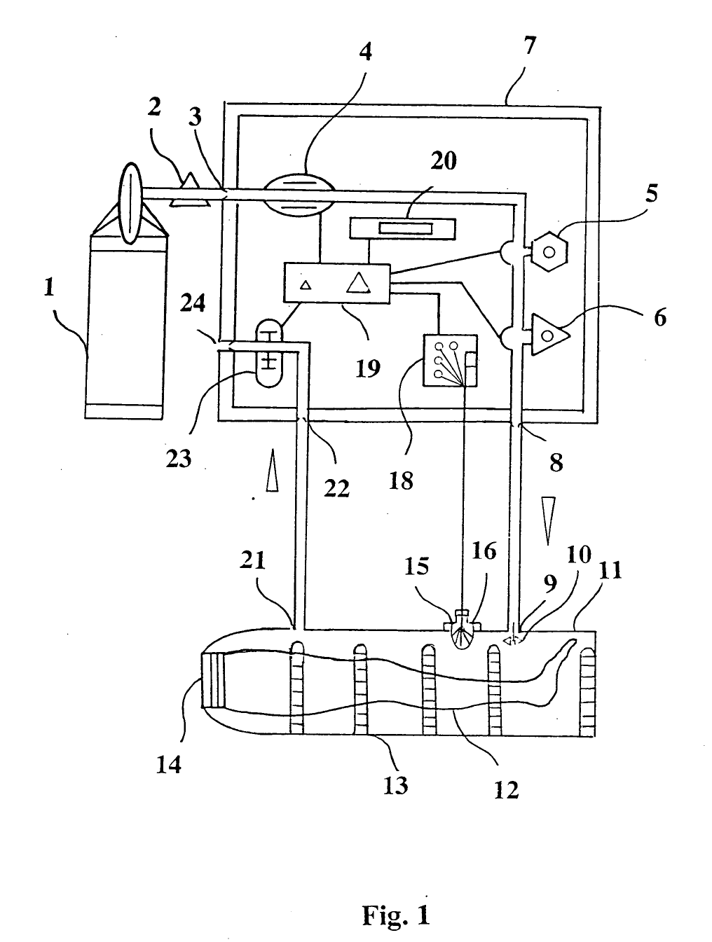

FIG. 1 shows schematically the configuration of apparatus according to an embodiment of the invention, and its use for the external O₃/O₂ treatment of an infected leg.

The medical grade oxygen tank (1) feeds oxygen through a regulator (2) and enters the ozone generator (7) through an intake valve (3).

A power unit (4) imparts electrical energy for converting the oxygen to ozone.

The O₂/O₃ mixture passes through a humidifier (5), then through a heater/cooler (6), exiting from the generator outflow valve (8) to enter the inlet (9) of the treatment envelope (11). An intake fan distributor (10) serves to homogenize the intra-envelope gas milieu.

The treatment envelope (11) encases the affected limb (12). Supporting ribs (13) hold the treatment envelope in a manner to prevent the sheath of the envelope from contacting the skin of the patient.

The envelope forms a hermetic seal (14) with the limb. This may be accomplished by means of a Velcro-type or adhesive-type seal.

The envelope contains an opening (15) through which is inserted a multi-sensor head (16) containing sensors for ozone concentration, oxygen concentration, temperature, humidity, and the presence of bacterial gases.

These sensors relay their signals to their respective analyzers, which are grouped in the analyzer unit (18).

All the above analyzers project their data to the microprocessor (19).

The microprocessor connects with the LCD (liquid crystal display) (20), to provide a digital readout of the data at hand.

The microprocessor, in addition, has reciprocal relationships with the power unit (4), the humidifier (5), the heater/cooler (6), and the analyzer unit (18).

Ozone/oxygen exits the treatment envelope through the envelope outlet valve (21) and enters the ozone generator (7) through its envelope effluent intake valve (22), and on to the ozone destructor (23) which de-energizes the remaining ozone, converting it to oxygen. This oxygen may safely exit the ozone generator through its exit valve (24).

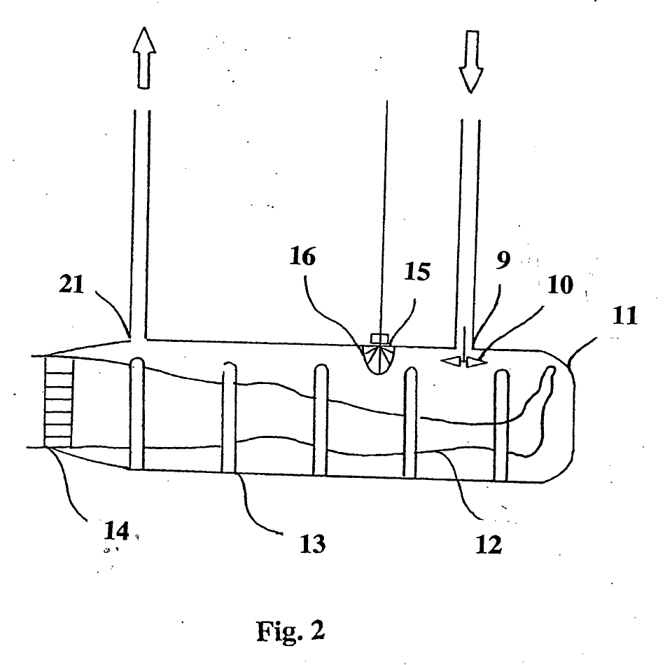

As seen in FIG. 2 the treatment envelope (11) encases the affected limb (12). The envelope hermetically seals the limb at (14) using a Velcro-type or adhesive-type fastener, for example.

Ribs (13) within the envelope keep it from collapsing. They prevent the envelope membrane (11) from touching the skin of the patient. The ribs shown are circumferential of the generally cylindrical envelope, but could take any other suitable configuration.

The envelope is provided with an entry port (15) for the easy insertion and removal of the multi-sensor head (16) from the ozone generator.

The multi-sensor head contains sensors including an ozone sensor, an oxygen sensor, a temperature sensor, a humidity sensor, and a bacterial gas sensor.

The ozone/oxygen mixture enters the envelope through inflow valve (9). A fan (10), incorporated in or near the inflow valve, works to homogenize the intra-envelope milieu. Gas exits the treatment envelope through its exit valve (21) for processing by the generator.

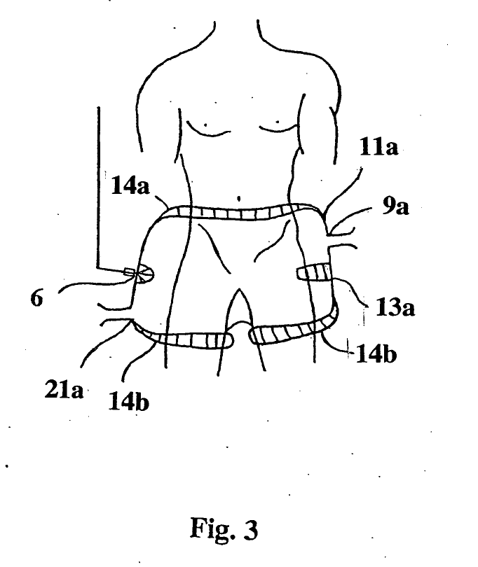

In FIG. 3, the treatment envelope (11a) shows a specialized configuration in the form of briefs. It is fitted with supporting ribs (13a), which keep the membrane of the briefs away from the patient's skin. The envelope hermetically seals the torso and legs by means of adhesive, or Velcro-type fasteners (14a, 14b).

Ozone/oxygen enters the envelope via its entry port (9a). The gas exits through the envelope exit port (21a), to join the ozone generator where it will be converted to oxygen.

The multi-sensor head (16) relays data about the intra-envelope ozone milieu to the analyzers and to the microprocessor in the generator.

Although the present invention has been described in relation to particular embodiments thereof, many other variations and modifications and other uses will become apparent to those skilled in the art. Therefore, the present invention is not limited by the specific disclosure herein.

What is claimed is:

1. An ozone/oxygen treatment system comprising:
an ozone generator for generating a predetermined ozone/oxygen mixture; and
a treatment envelope connected to said ozone generator for receiving and applying said ozone/oxygen mixture to a predetermined portion of a patient's body,
said treatment envelope being sized and shaped for enclosing said predetermined body portion and having a structure enabling said treatment envelope to enclose without touching said body portion.

2. The system of claim 1, wherein a space surrounding said body portion within said treatment envelope is not more than approximately 10-15% of the total volume of said treatment envelope.

3. The system of claim 1, further comprising a fan disposed within said treatment envelope.

4. An ozone/oxygen treatment system comprising:
an ozone generator for generating a predetermined ozone/oxygen mixture; and
a treatment envelope connected to said ozone generator for receiving and applying said ozone/oxygen mixture to a predetermined portion of a patient's body,
further comprising a sensor disposed in said treatment envelope for sensing at least one of ozone concentration, temperature, humidity and bacterial gases.

5. The system of claim 4, further comprising an opening in said treatment envelope for removably receiving said sensor.

6. The system of claim 5, wherein said sensor is comprised in a multi-sensor head for sensing more than one of said ozone concentration, temperature, humidity and bacterial gases.

7. The system of claim 4, wherein said sensor is comprised in a multi-sensor head for sensing more than one of said ozone concentration, temperature, humidity and bacterial gases.

8. The system of claim 4, further comprising a supporting structure in said treatment envelope for enabling said treatment envelope to enclose without coming into contact with said body part.

9. The system of claim 8, wherein said supporting structure comprises an ozone-resistant material.

10. The system of claim 9, wherein said treatment envelope has a generally curved surface and said supporting structure comprises at least one rib defining the curve of said surface.

11. The system of claim 4, further comprising a fan within said treatment envelope.

12. The system of claim 4, further comprising a control unit receiving data from said sensor and in response to said data, automatically controlling said ozone generator to maintain said ozone concentration at a predetermined range.

13. The system of claim 12, further comprising apparatus for supplying at least one of heat and humidity to said treatment envelope,
said apparatus being automatically controlled by said control unit to maintain said heat and/or humidity at a predetermined range.

14. The system of claim 12, wherein said control unit is operative for controlling said ozone concentration as a function of time.

15. The system of claim 12, wherein said control unit is operative for delivering pure oxygen to said treatment envelope.

16. A method of treating a predetermined body part with an ozone/oxygen mixture, comprising the steps of:
generating a predetermined ozone/oxygen mixture; and
supplying said ozone/oxygen mixture to a treatment envelope enclosing said predetermined body part,
said treatment envelope being sized and shaped for enclosing said predetermined body part and having a structure enabling said treatment envelope to enclose without touching said body part.

17. The method of claim 16, wherein a space surrounding said body portion within said treatment envelope is not more than approximately 10-15% of the total volume of said treatment envelope.

18. The method of claim 16, further comprising the step of circulating said ozone/oxygen mixture within said treatment envelope.

19. A method of treating a predetermined body part with an ozone/oxygen mixture, comprising the steps of:
generating a predetermined ozone/oxygen mixture;
supplying said ozone/oxygen mixture to a treatment envelope enclosing said predetermined body part; and
sensing at least one of ozone concentration, temperature, humidity and bacterial gases within said treatment envelope.

20. The method of claim 19, further comprising the step of removably receiving a sensor through an opening in said treatment envelope.

21. The method of claim 20, further comprising the step of providing a multi-sensor head for sensing more than one of said ozone concentration, temperature, humidity and bacterial gases.

22. The method of claim 19, further comprising the step of providing a multi-sensor head for sensing more than one of said ozone concentration, temperature, humidity and bacterial gases.

23. The method of claim 19, further comprising the step of disposing a supporting structure in said treatment envelope for enabling said treatment envelope to enclose without coming into contact with said body part.

24. The method of claim 23, wherein said supporting structure comprises an ozone-resistant material.

25. The method of claim 24, wherein said treatment envelope has a generally curved surface and said supporting structure comprises at least one rib defining the curve of said surface.

26. The method of claim 19, further comprising the step of circulating said ozone/oxygen mixture within said treatment envelope.

27. The method of claim 19, further comprising the step of receiving data from said sensor, and in response to said data, automatically controlling said ozone generator to maintain said ozone concentration at a predetermined range.

28. The method of claim 27, further comprising the steps of supplying at least one of heat and humidity to said treatment envelope, and maintaining said heat and/or humidity at a predetermined range.

29. The method of claim 27, further comprising the step of controlling said ozone concentration as a function of time.

30. The method of claim 27, further comprising the step of delivering pure oxygen to said treatment envelope.
